(19) United States
(12) Patent Application Publication
(10) Pub. No.: US 2010/0331867 A1
(43) Pub. Date: Dec. 30, 2010

(54) APPARATUS AND METHOD FOR DERMAL INCISION

(76) Inventor: Joseph Giovannoli, Park Ridge, NJ (US)

Correspondence Address: Kaplan Gilman & Pergament LLP, 1480 Route 9 North, Woodbridge, NJ, 07095, US

(21) Appl. No.: 12/824,836

(22) Filed: Jun. 28, 2010

Related U.S. Application Data
(60) Provisional application No. 61/220,653, filed on Jun. 26, 2009.

(51) Int. Cl. A61B 17/3209

(52) U.S. Cl. 606/167

(57) ABSTRACT

An incision instrument may include a deformable hollow blade that is acted upon by two or more clamping arms that deform the blade into an elliptical cross-sectional shape, thereby enabling the incision instrument to make controlled elliptical incisions.

This application claims the benefit of U.S. Provisional Application Ser. No. 61/220,653, filed Jun. 26, 2009, entitled “Apparatus and Method for Dermal Incision” the entire disclosure of which is incorporated by reference herein.

### BACKGROUND OF THE INVENTION

This invention relates in general to apparatus for making tissue incisions and in particular to adjusting one or more parameters of the tissue incision and the tissue incision process.

When removing a tissue biopsy specimen or a mole or other tissue lesion, it is known to cut the tissue along a closed line completely penetrating the tissue and to then remove the specimen by further sharp and blunt dissection. Thereafter, the resulting wound may be appropriately closed and protected against infection. It is also known to use punch-type instruments having sharp blades to accurately cut closed lines in the tissue. A surgeon can hold and manipulate such instruments using one hand, and use his other hand to stretch the tissue at the site of the incision.

Incisions of elliptical shape, or substantially elliptical shape, are generally considered to have beneficial characteristics. Elliptical incisions may enable more medically effective and more aesthetically pleasing healing of the tissue after the incision procedure is over.

However, attempting to create an incision having a consistent elliptical pattern using free-hand cutting incurs several difficulties. Suitably stretching the tissue properly for the incision is difficult when cutting by hand. Moreover, maintaining a consistent, symmetric cutting pattern is difficult when cutting by hand. Accordingly, the use of a punch type instrument may be preferable to free-hand cutting.

To date, most punch type incision devices have been circular. Thus, creating an elliptical incision may require a surgeon to modify the intended use of the instrument to achieve an elliptical cut. Such improved, unintended use may lead to inconsistent results that depend on the skill of the surgeon and the characteristics of the incision device used.

Accordingly, there is a need in the art for an improved system and method for enabling a surgeon to generate an elliptical incision in a patient's tissue.

### SUMMARY OF THE INVENTION

According to one aspect, the invention is directed to an incision instrument that may include a handle having a proximal end and a distal end; a mount moveably engaged with the handle, along a longitudinal axis of the handle; a tubular blade surrounding an enclosed space, the blade having a proximal end coupled to the handle and a second, distal end extending into free space, and configured for cutting tissue; and a clamp coupled to the mount and operable to impart a compressive force on an external surface of the blade, the force being operable to deform the blade. Preferably, the clamp and the blade are configured such that advancement of the mount and clamp along the longitudinal axis of the handle causes progressively increasing deformation of the blade. Preferably, movement of the mount is operable to control an amount of geometric distortion of the blade. Preferably, the mount is slidably engaged with the handle. Preferably, the clamp is springingly biased so as to impart the compressive force on the external surface of the blade.

Preferably, the clamp is slidably engaged with the blade, and wherein advancement of the mount and clamp outward along the longitudinal axis of the handle causes the clamp to apply the compressive force to the external surface of blade at a progressively greater distance from the proximal end of the blade. Preferably, the greater the distance between (a) the point at which the clamp applies compressive force to the blade; and (b) a point at which the blade is coupled to the handle, the more the applied force deforms the blade. Preferably, the applied force deforms the cross-sectional shape of the blade into an elliptical shape. Preferably, the clamp comprises: at least two clamp arms that are springingly biased so as to apply the compressive force to the external surface of the blade.

According to another aspect, the invention is directed to an incision instrument that may include a handle having a proximal end and a distal end; a mount assembly slidably engaged with the handle, along a longitudinal axis of the handle; and a tubular blade surrounding an enclosed space, the blade having a proximal end coupled to the handle and a second, distal end extending into free space and configured for cutting tissue; wherein the mount assembly is constructed to compress the blade along one dimension upon being advanced toward the distal end of the handle. Preferably, the blade has a substantially circular cross-sectional shape when the mount assembly is near the proximal end of the handle, and wherein the advancement of the mount assembly toward the distal end of the handle causes the cross-sectional shape of the blade to deform into an elliptical shape. Preferably, the mount assembly and handle cooperate to enable a user to continuously vary a shape of a cutting edge of the blade through advancement of the mount toward the distal end of the handle. Preferably, the mount assembly includes a mount slidably engaging the handle; and a clamp disposed between the mount and the blade and operable to impart force to the blade.

According to yet another aspect, the invention is directed to a method that may include providing an incision instrument having a handle, a mount assembly slidably engaging the handle, and a tubular blade coupled at a proximal end thereof to the handle, the tubular blade having a distal cutting end that has a substantially circular cross section; and adjusting the cross-sectional shape of the distal cutting end of the blade using the mount assembly. Preferably, the step of adjusting includes moving the mount assembly along the longitudinal axis of the handle.

Other aspects, features, advantages, etc. will become apparent to one skilled in the art when the description of the preferred embodiments of the invention herein is taken in conjunction with the accompanying drawings.

### BRIEF DESCRIPTION OF THE DRAWINGS

For the purposes of illustrating the various aspects of the invention, there are shown in the drawings forms that are presently preferred, it being understood, however, that the invention is not limited to the precise arrangements and instrumentalities shown.

FIG. 1 is an elevational view of an incision instrument in accordance with an embodiment of the present invention;

FIG. 1A is a sectional view of an end of a blade forming part of the incision instrument of FIG. 1;

FIG. 2 is an elevational view of a mount and clamp for use with an incision instrument in accordance with an embodiment of the present invention;

FIG. 2A is a sectional view of an end of the mount and clamp shown in FIG. 2;

FIG. 3 is an elevational view of an incision instrument including a mount and clamp in accordance with an embodiment of the present invention;

FIG. 3A is a sectional view of the clamp impinging on a portion of the handle of the incision instrument of FIG. 3;

FIG. 4 is an elevational view of an incision instrument showing deformation of a cutting blade in accordance with an embodiment of the present invention;

FIG. 4A is a sectional view of the clamp deforming the cross-sectional shape of the blade of the incision instrument of FIG. 4;

FIG. 5 is an exploded elevational view of an incision instrument in accordance with another embodiment of the invention;

FIG. 5A is a sectional view of a blade forming part of the incision instrument of FIG. 5;

FIG. 6 is an elevational view of the incision instrument of FIG. 5 showing an initial insertion of clamp arms into the blade;

FIG. 6A is a sectional view of the blade and clamp arms of the incision instrument of FIGS. 5-6;

FIG. 7 is an elevational view of the incision instrument of FIGS. 5-6 with the clamp more deeply inserted into the blade; and

FIG. 7A is a sectional view of the embodiment of FIG. 7.

### DETAILED DESCRIPTION OF THE PREFERRED EMBODIMENTS

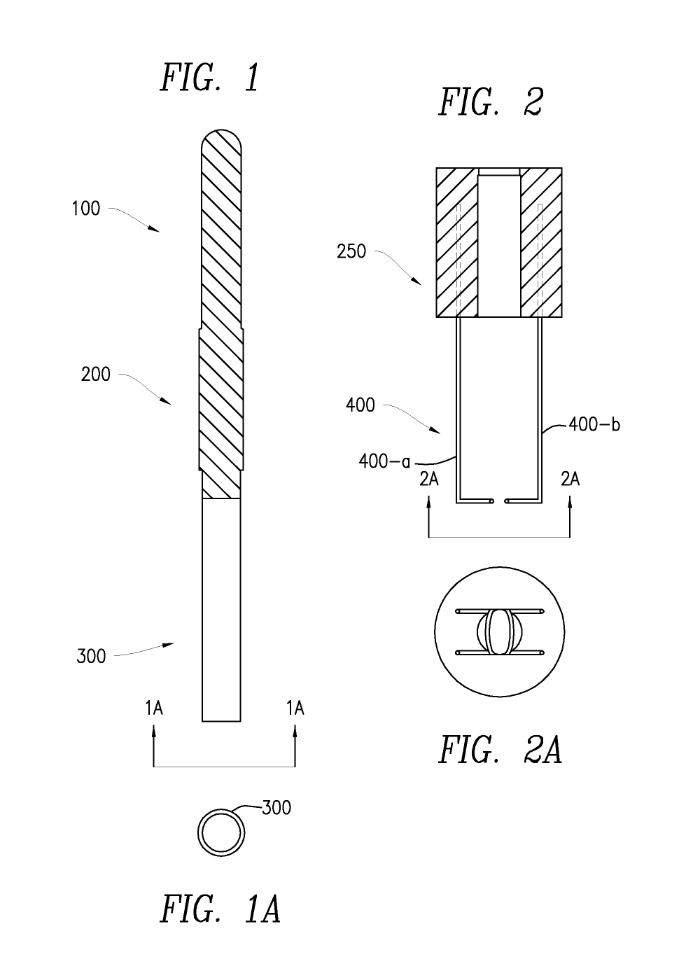

Reference is made to FIGS. 1-4 in the following. Incision instrument 100 may include handle 200 and blade 300. FIGS. 1-4 show a single substantially circular blade 300. However, two or more blades may be employed. Blade 300 may be assembled to handle 200. Blade 300 may be hollow and have a circular cross section. However, in other embodiments, blades 300 having other configurations may be employed. Otherwise stated, blade 300 may be a tubular blade surrounding an enclosed space with a proximal end coupled to the handle 200 and a second, distal end extending into free space (that is, unconstrained by any mechanical coupling), wherein the distal end is preferably configured for cutting tissue, such as by being suitably sharpened and/or deburred. The tissue referred to above may include, but is not limited to: skin, fat, internal organ material, among other bodily materials suitable for being cut with a sharp-edged instrument.

Incision instrument 100 may further include mount 250 and clamp 400. Mount 250 may be attached to, or otherwise fixed laterally with respect to, handle 200. Mount 250 is preferably moveable, along a longitudinal axis of both mount 250 and handle 200, with respect to handle 200 and blade 300. Incision instrument 100 may also be described as having a handle 200, a mount assembly, and a blade 300, where the mount assembly includes the mount 250 and the blade 300. However, the mount assembly of the present invention is not limited having mount 250 and blade 300. Indeed, other configurations of incision instrument 100 operable to cause a tubular blade 300 to adopt an elliptical cross-sectional shape, upon advancement of the mount assembly along the longitudinal axis of the handle 200, may be practiced, and all such variations are intended to be included within the scope of the present invention.

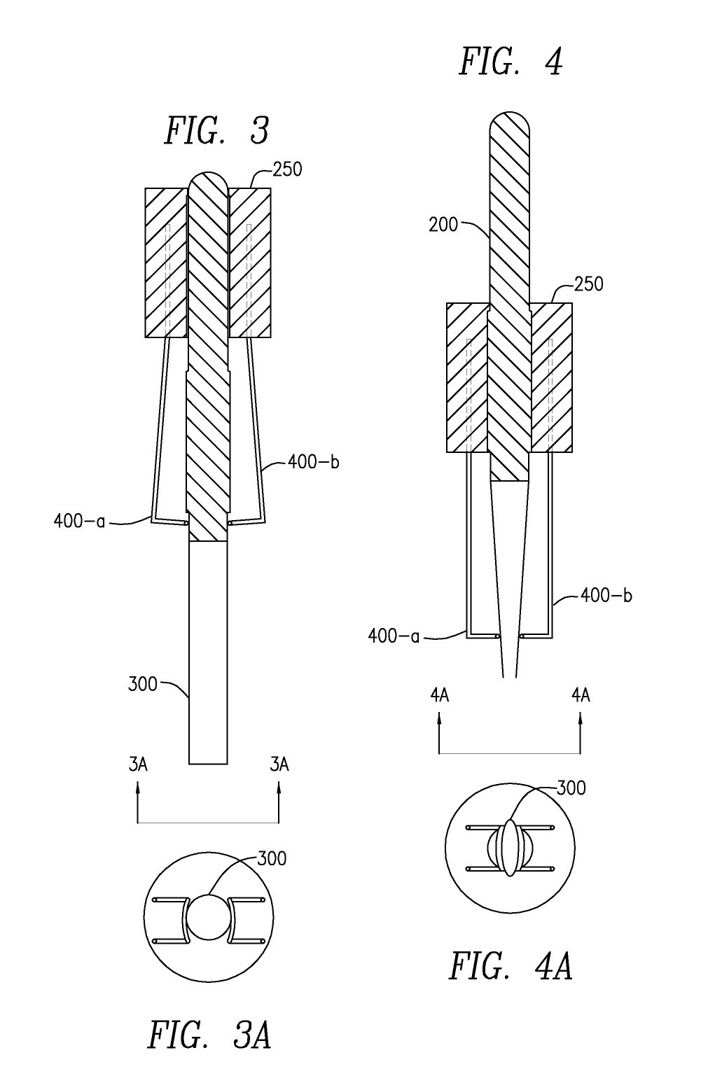

A first, proximal end of clamp 400 may be connected to mount 250, and a second end of clamp 400 may be positioned and configured to impinge on the outer diameter of blade 300 to deform the cross-sectional geometry of blade 300 into a desired shape, as shown in FIG. 4A. Clamp 400 may include clamp arms 400-a and 400-b. While two clamp arms are shown, fewer or more than two clamp arms may be employed. Clamp arms 400-a, 400-b may include contact surfaces for engaging the outer diameter of blade 300, as shown in FIG. 3A. These clamp-arm contact surfaces may be flat or curved depending on the needs of a given application. The proximal ends (the top ends in the views of FIGS. 2-4) of clamp arms 400-a and 400-b may be anchored at a pivot point within mount 250. Clamp arms 400-a and 400-b may be mounted such that the distal ends thereof, when at rest, are closer together than the diameter of blade 300. In this manner, clamp arms 400-a and 400-b can be mounted so as to always a compressive force on the external surface of blade 300 when in contact therewith.

Incision instrument 100 may be beneficially employed to cause a blade 300 that is circular without the imposition of external force to deform into a substantially elliptical shape to enable use of instrument 100 to make substantially elliptical incisions into human tissue or other material to be cut by instrument 100. While substantially elliptical shapes are discussed herein, blade 300 may be forced into occupying other shapes as well.

A discussion of the operation of instrument 100 is conducted with reference to FIG. 3. Mount 250 is shown positioned near the top of handle 200. Clamp arms 400-a, 400-b may be spring loaded or otherwise biased so as to push inward against a portion of handle 200, in the view of FIG. 3. Spring loading may be provided by a range of possible devices including linear and/or rotational springs. Alternatively, manual adjustment of the position of clamp arms 400-a and 400-b may be employed to achieve a desired force level on arms 400-a and 400-b and a corresponding desired degree of geometric distortion of blade 300.

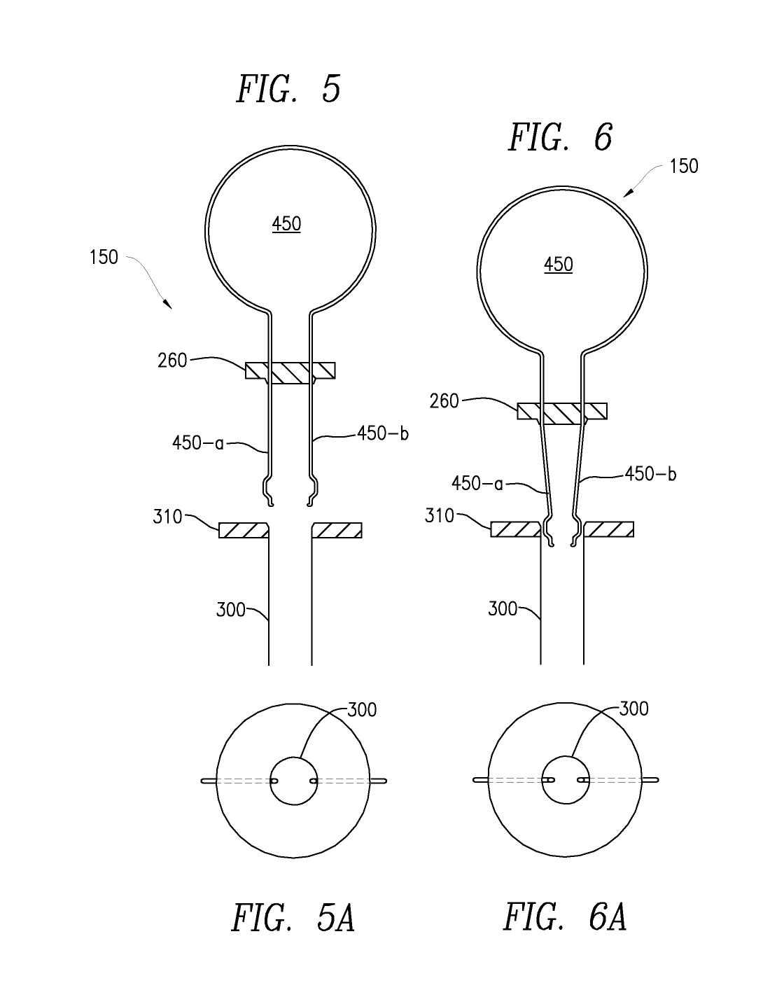

In this section the balance of forces is discussed in connection with the embodiment shown in FIG. 4. However, the principles discussed in this section are equally applicable to the embodiment of FIGS. 5-7. As clamp arms 400-a and 400-b of clamp 400 apply compressive force to blade 300, the resistance to deformation of blade 300 counteracts the compressive force of the clamp 400. This preferably establishes a balance of forces between the expansive spring force of blade 300 and the compressive force of clamp 400. Thus, instead of having blade 300 assume a fixed shape dictated by the shape of a guide forming part of a housing or other structure, the spring constants of blade 300 and clamp 400, and the position of mount 250 and clamp 400 along the longitudinal axis of handle 200 combine to determine the size and shape of the cutting edge of blade 300, which will generally be elliptical upon the application of force on blade 300 by clamp 400. Further, the resilience of blade 300 (i.e. the ability of blade 300 to restore its natural shape upon the reduction and/or removal of compressive force from clamp 400) preferably enables blade 300 to self-adjust outward as the force from clamp 400 is reduced. In the case where the force from clamp 400 is completely removed from blade 300, blade 300 preferably fully restores its original shape, thereby providing a substantially circular cutting edge, as shown in FIG. 3 and FIG. 3A.

To prepare instrument 100 for cutting, mount 250 may be moved along the longitudinal axis of handle 200 (in the downward direction in the view of FIGS. 3-4). As mount 250 is moved along handle 200, clamp arms 400-a and 400-b engage blade 300 and impart force thereto. As mount 250 is advanced toward a most advanced position (shown in FIG. 4), clamp arms 400-a and 400-b impart a force on blade 300 thereby causing blade 300 to deform. The resulting deformation of blade 300 is shown in FIG. 4A. As expected, the cross-sectional profile of blade 300 is made narrower in the dimension in which the force is applied and is made longer in dimension perpendicular to the direction in which force is applied, thus providing the substantially elliptically shaped blade 300 cross section shown in FIG. 4A. In this manner, the advancement of mount 250 toward the distal end of handle 200 (the downward direction in the view of FIG. 4) causes a progressively increasing deformation of the shape of blade 300, as shown in FIG. 4A. Thus, the embodiment of FIG. 4 preferably enable a user of incision instrument 100 to continuously vary a shape of the cutting edge (the bottom edge in the view of FIG. 4) of blade 300 by advancing the mount 250 outward (toward the distal end of handle 200) along the longitudinal axis of handle 200. It is noted that blade 300 is not limited to being used with the degree of deformation shown in FIGS. 4 and 4A. Alternatively, a user may elect to advance mount 250 along only a portion of the permissible displacement along handle 200 in order to cause blade 300 to assume shape in between the highly elliptical cross-sectional shape shown in FIG. 4A and the substantially circular shape shown in FIG. 3A.

Thereafter, blade 300, in its substantially elliptically deformed condition may be used to cut tissue by appropriately moving incision instrument 100 toward a tissue surface into which an incision is desired. Incision instrument 100 may be used as a single punch to form a single substantially elliptical incision. Alternatively, a one-dimensional or two-dimensional array of blades 300 may be assembled onto a common base structure and used to produce a corresponding array of incisions on a patient's tissue. In this manner, incision instrument 100 may be used to produce an array of incisions having a pre-determined geometrical arrangement. In one embodiment, the geometric arrangement of the array of blades may itself be substantially elliptical, thereby operating to provide a substantially elliptically shaped array of incisions on a patient's tissue. In other embodiments, other geometric distributions of blades 300 and resulting incisions may be employed. Enhanced cutting action may be obtained as blade 300 is pressed into a patient's tissue by rotating and/or counter-rotating blade 300 while holding the axis of substantially elliptical blade 300 in a fixed orientation with respect to the tissue being cut.

FIG. 5 is an exploded elevational view of an incision instrument 150 in accordance with another embodiment of the invention. Incision instrument 150 may include clamp 450 having clamp arms 450-a and 450-b, clamp brace 260, blade 300, and blade flange 310.

FIG. 5 shows incision instrument 150 in a disassembled state. Correspondingly, sectional view FIG. 5A shows blade 300 having an undisturbed circular cross-sectional configuration. In this embodiment as in the first embodiment described above, blade 300, is preferably hollow.

FIG. 6 shows expansion clamp 450 at an initial stage of insertion into blade 300. More specifically, clamp arms 450-a and 450-b are shown entering the hollow interior of blade 300. At the stage of insertion shown in FIG. 6, the cross-sectional geometry of blade 300 has not been significantly deformed from its at-rest condition of having a circular cross-sectional geometry, as shown in FIG. 6A.

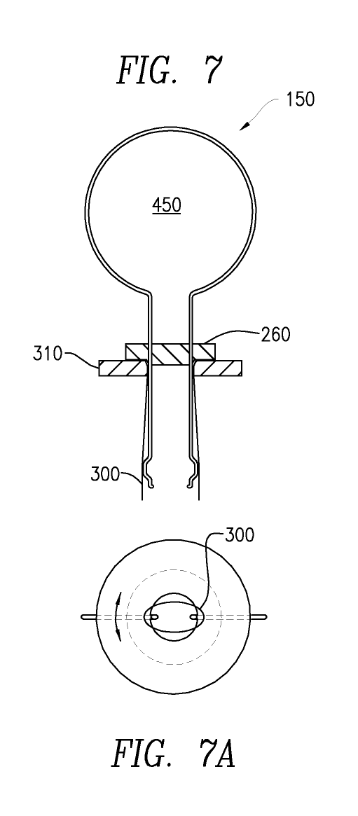

FIG. 7 shows expansion clamp 450 fully inserted into blade 300, as indicated by the contact between clamp mount 260 and blade flange 310. Clamp arms 450-a and 450-b are shown deforming the cross-sectional geometry of blade 300 into a substantially elliptical shape as shown in FIG. 7A.

As with the embodiment of FIGS. 1-4, incision instrument 150 may include a single blade 300. Alternatively, incision instrument 150 may include a plurality of blades 300 that may be arranged in a one or two-dimensional array. For instance, a plurality of blades 300 may be arranged in a two-dimensional array that is itself substantially elliptically shaped.

### Methods of Use:

Attention is now directed to preferred methods of using the above described embodiments to create incisions. Desired cutting action of blade 300 against the tissue of a patient may be achieved by holding the blade deformation means (such as clamps 400 and/or 450) fixed in relation to a tissue surface, and rotating (either in one direction or using back-and-forth motion) handle 200 and blade 300 with respect to the stationary tissue. The rotation of the handle 200 and blade 300 may be done manually. Alternatively, a motor can be used to provide the rotation. In this manner, the cutting edge of blade 300 may be moved so as to provide a cutting action against a tissue surface while remaining within a substantially elliptical cutting path. Alternatively, the incision instruments 100, 150 described herein may be merely pressed against the tissue to cut a desired incision.

Attention is directed to FIGS. 5-7 in connection with the application of the cutting method to the embodiment including the expansion clamp 450. To achieve the desired cutting action, in one embodiment, a user may manually hold the round portion of expansion clamp 450 fixed in relation to the surface of tissue to be cut and place blade 300 up against the tissue surface. Thereafter, the user may turn blade 300 to cut the tissue, while holding expansion clamp 450 fixed in relation to the tissue surface. In this manner the elliptical cutting surface of blade 300 (see FIG. 7A) moves along a cutting path precisely corresponding to its own elliptical shape. In this manner, embodiments of the present invention avoid scraping of the skin by moving the narrow ends of the elliptically shaped (which may also be referred to as oval shaped) blade 300 about a simple circular axis. This cutting action preferably accurately creates an elliptically shaped cut in the tissue that closely corresponds to the elliptical shape of blade 300. In alternative embodiments, movement of the blade may be provided using motorized or other powered means, instead of moving blade 300 manually.

The above-described “saw-like” cutting action provides a desirable benefit over prior art non-circular punches, and punches of any geometry for that matter, that can only be pressed into the tissue but which do not enable the blade to moved along the tissue in a “sawing” action as described for the embodiments herein.

For any of the above structural and/or method embodiments, the tissue may be stretched prior to commending an incision procedure. Alternatively, the tissue may be left unstretched.

The device can be used with a motor so that the flexible blade spins and the proper portions are bent to conform to the substantially elliptical shape as the blade rotates. Additionally, by holding the deforming means in the same orientation, while the flexible blade rotates, an incision is formed in a substantially elliptical shape.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An incision instrument comprising:
a handle having a proximal end and a distal end;
a mount moveably engaged with the handle, along a longitudinal axis of the handle;
a tubular blade surrounding an enclosed space, the blade having a proximal end coupled to the handle and a second, distal end extending into free space, and configured for cutting tissue; and
a clamp coupled to the mount and operable to impart a compressive force on an external surface of the blade, the force being operable to deform the blade.

2. The incision instrument of claim 1 wherein the clamp and the blade are configured such that advancement of the mount and clamp along the longitudinal axis of the handle causes progressively increasing deformation of the blade.

3. The incision instrument of claim 1 wherein movement of the mount is operable to control an amount of geometric distortion of the blade.

4. The incision instrument of claim 1 wherein the mount is slidably engaged with the handle.

5. The incision instrument of claim 4 wherein the clamp is springingly biased so as to impart the compressive force on the external surface of the blade.

6. The incision instrument of claim 5 wherein the clamp is slidably engaged with the blade, and wherein advancement of the mount and clamp outward along the longitudinal axis of the handle causes the clamp to apply the compressive force to the external surface of blade at a progressively greater distance from the proximal end of the blade.

7. The incision instrument of claim 6 wherein the greater the distance between (a) the point at which the clamp applies compressive force to the blade; and (b) a point at which the blade is coupled to the handle, the more the applied force deforms the blade.

8. The incision instrument of claim 7 wherein the applied force deforms the cross-sectional shape of the blade into an elliptical shape.

9. The incision instrument of claim 1 wherein the clamp comprises: at least two clamp arms that are springingly biased so as to apply the compressive force to the external surface of the blade.

10. An incision instrument comprising:
a handle having a proximal end and a distal end;
a mount assembly slidably engaged with the handle, along a longitudinal axis of the handle; and
a tubular blade surrounding an enclosed space, the blade having a proximal end coupled to the handle and a second, distal end extending into free space and configured for cutting tissue;
wherein the mount assembly is constructed to compress the blade along one dimension upon being advanced toward the distal end of the handle.

11. The incision instrument of claim 10 wherein the blade has a substantially circular cross-sectional shape when the mount assembly is near the proximal end of the handle, and wherein the advancement of the mount assembly toward the distal end of the handle causes the cross-sectional shape of the blade to deform into an elliptical shape.

12. The incision instrument of claim 10 wherein the mount assembly and handle cooperate to enable a user to continuously vary a shape of a cutting edge of the blade through advancement of the mount toward the distal end of the handle.

13. The incision instrument of claim 10 wherein the mount assembly comprises:
a mount slidably engaging the handle; and
a clamp disposed between the mount and the blade and operable to impart force to the blade.

14. A method, comprising:
providing an incision instrument having a handle, a mount assembly slidably engaging the handle, and a tubular blade coupled at a proximal end thereof to the handle, the tubular blade having a distal cutting end that has a substantially circular cross section; and
adjusting the cross-sectional shape of the distal cutting end of the blade using the mount assembly.

15. The method of claim 14 wherein the step of adjusting comprises:
moving the mount assembly along the longitudinal axis of the handle.
